(19) United States
(12) Patent Application Publication
(10) Pub. No.: US 2014/0286927 A1
(43) Pub. Date: Sep. 25, 2014

(54) METHOD FOR THE TREATMENT OF ULCERATIVE COLITIS IN PATIENTS REFRACTORY TO STEROID THERAPY USING LEACHED LIGAND FIBRIN STABILIZING COMPOSITION

(71) Applicant: Peter Edward Smith, Spring City, PA (US)

(72) Inventor: Peter Edward Smith, Spring City, PA (US)

(21) Appl. No.: 13/526,690

(22) Filed: Jun. 19, 2012

(51) Int. Cl. A61K 38/45; A61K 45/06

(52) U.S. Cl. CPC A61K 38/45; A61K 45/06
USPC 424/94.5; 562/597; 564/511; 546/257; 546/88; 568/412; 549/352; 540/469; 540/474; 568/17; 564/512; 423/462; 423/511; 423/366; 423/395; 423/579; 423/580.1; 423/519.2; 423/352; 423/385; 423/418.2

(57) ABSTRACT

A engineered composition and method of delivery of said composition providing effective therapy for the treatment of ulcerative colitis, and Crohn's disease.

### FIELD OF INVENTION

The present disclosures relates to providing effective therapy for the treatment of Inflammatory Bowel Disease (Ulcerative Colitis, and Crohn's Disease).

### BACKGROUND OF THE INVENTION

Currently there is no known cause or cure for Inflammatory Bowel Disease. Patients with severe attacks are typically hospitalized and closely monitored. After failing three to five days of intravenous corticosteroids, patients are considered for either intravenous Cyclosporine (2 mg/kg per day), or Infliximab (5 mg/kg) and/or surgical colectomy.

Ulcerative colitis (UC), Crohn's diseases are Inflammatory Bowel Diseases (IBD) characterized by recurring episodes of inflammation, unrelated to intestinal infection, in the mucosal layer of the large bowel. The inflammation involves the rectum and may extend proximally in a symmetrical, circumferential, and uninterrupted pattern to involve parts or all of the large intestine. The hallmark clinical symptom is bloody diarrhea often with prominent symptoms of rectal urgency and tenesmus. The clinical course is marked by exacerbations and remissions, which may occur spontaneously or in response to the treatment changes or intercurrent illnesses. Oral or rectal amino salicylates, specifically mesalamine, are considered to be first line therapy for patients with mild-moderate disease. About half of patients treated with steroids will achieve complete remission in 4 weeks, but about one quarter of those patients become steroid dependent. Azathioprine/6-mercaptopurine (AZA/6-MP) is used for such patients, but their onset of action is 3-6 months, limiting its use for acute induction of remission. Cyclosporine has also been used for severe diseases. An anti-TNFα monoclonal antibody, infliximab (Remicade), has been shown effective in patients with moderate disease, producing clinical responses and mucosal healing in about 65-67% of patients. For ulcerative colitis patients with severe colitis refractory to maximal oral therapy, admission to the hospital and treatment by intravenous steroids are required.

Common symptoms of ulcerative colitis include rectal bleeding and diarrhea, but there is a wide range of symptoms among patients with this disease. Variability of symptoms reflects differences in the extent of disease (the amount of the colon and rectum that are inflamed) and the intensity of inflammation. Generally, patients with inflammation confined to the rectum, and a short segment of the colon adjacent to the rectum, have milder symptoms and a better prognosis than patients with more widespread inflammation of the colon. The different types of ulcerative colitis are classified according to the location and extent of inflammation.

In the disease states representing, FXIII (Factor XIII) deficiency is associated with severe bleeding, along with spontaneous bleeding and poor wound healing has been also noted. During wound healing, proteins play an integral role in forming a matrix of cross-linkages of surrounding platelets and other complexes of growth factors to include FXIII to form the final blood clot or scar. In this absence, the formation of the scar is delayed and its mechanical properties are impaired. Impairment of epithelialization of the lining of the colon may be compromised by the lack of FXIII available to attach to newly formed connective tissue before reconstruction of the basement membrane during reconstitution of the epithelium.

The use of fibrin stabilizing compounds to help with ulcerative colitis is well known as well as the inherent limitations of single compound solutions, leading to regulatory rejections by the US FDA among others.

Fibrin stabilization compounds have been limited to Factor XIII or recombinant solutions that are not designed from a functional perspective.

Urabe et al. teach in U.S. Pat. No. 5,378,687 about the use of human blood coagulation FXIII for the treatment of Ulceration Colitis in four representative cases. It is vague with respect with duration of treatment and focuses solely on the impact of the naturally occurring FXIII element and does not include the impact of other chemical and biological elements used in conjunction with FXIII in the healing and administration process, thus limiting the applicability of the treatment to a certain class of patient.

Fox teaches in U.S. Pat. No. 6,887,695 the use of recombinant Abstract: Method patent identifying a polypeptide responsible for the crosslinking of large proteins and the incorporation of small primary amines into proteins. Transglutaminases are believed to be widely distributed in nature, since these crosslinks are found in both prokaryotic and eukaryotic cells.

Ohrstrom teaches in US Patent Application 20070122384 treating inflammatory bowel disease by administering interferon beta in conjunction with FXIII to a mammal afflicted with the disease.

There is a need for a FXIII based solution that is more universally applicable, non-toxic and effective for treatment of IBDs.

### OBJECT AND SUMMARY OF THE INVENTION

A method for treating ulcerative colitis by administering fibrin stabilizing compound engineered has been demonstrated to be an effective alternative for patients who desire to avoid surgery and adjuvant immunotherapy, which may expose patients to higher potential rate of side effects to include significant toxicity, nephrotoxicity, infection and seizures.

### DETAILED DESCRIPTION OF THE INVENTION

FXIII (Factor XIII) is a zymogen of a class of enzymes known as transglutaminases that can form lysine crosslinks between two polypeptide chains. It has been shown that FXIII of plasma is a tetramer consisting, of two types of subunits (A2B2), while in cellular FXIII only the potentially active A subunits are present. It has been noted that FXIII plays an important role in the regulation of fibrinolysis, which is a critical aspect of the blood coagulation cascade. Also, the cellular form of FXIII is present in monocytes and different types of macrophages, including tissue macrophages. It has become clear that FXIII, in addition to a clotting Factor, is also a cellular enzyme with implications far beyond the clotting system.

This invention describes a composition and the use of a composition that is engineered to enhance the performance of fibrin stabilizers for inflammatory bowel diseases by integrating one or more ligands that bind FXIII proteins to sections of the bowel heretofore incapable of receiving said FXIII, whether naturally extracted or synthesized. We refer to this class of composition as Leached Ligand Fibrin Stabilizing (LLFS) composition.

In the thirty-three patients evaluated, 10/18 (55.6%) patients under FXIII and 4/15 (26.7%) under placebo achieved remission on day 42 demonstrating a clear trend towards significance. Also, the median CAI dropped in the subjects treated with FXIII from 12 to 6 on day 14 and further down to 2 on day 42 as compared to placebo (11 at start, 5 on day 14, 6 on day 42).

Ligands are used in Affinity Chromatography due to absorbent ability to specifically recognize the protein of interest. When a mixture of proteins is passed through the column, only those few that bind strongly to the ligand stick, while the others pass through the column. Covalent interaction occurs allowing the protein of interest to adhere to the column material. Protein of interest is eluted with a buffer containing free ligand, which competes with the column ligand to bind to the protein, and protein washes off however bound or leached ligand will also pass within the solution.

Based on the quantitative analysis of a composite index score for ulcerative colitis it is claimed that a response to treatment in patients with the disease can be measured in as few as three to four clays or as long as 42 days. The colitis activity index (CAI) is a quantitative measurement that incorporates a minimum of the following:

Inflammation in the colon based on colonoscopy

Diarrhea

Abdominal pain and cramping

Bloody stools

In another embodiment, the material elution process is used to engineer a fibrin-stabilizing composition: Since the early nineteen seventies, affinity chromatography has been investigated for its use in the purification of many biomolecules. These methods present advantages of a several thousand-fold enrichment of the target protein out of large volumes of crude starting materials, combined with high recoveries as well as the targeted separation of active and inactive material of denatured or functionally different forms. Roque et at. (Roque et al., 2007) and Low et al. (Low et al., 2007) have written reviews of the development of affinity ligands used for plasma purification. The removal of the target molecules is very efficient; more than 95% can easily be achieved. Non-specific binding of other proteins is possible due to the low affinity effect of the ligand.to the molecule of interest. One of the main disadvantages affinity resins is ligand leaching. As much as 1.45 μg/mL and 1.76 μg/mL ligand were detected in the final container concentrated to a 10% protein solution.

Fibrin Stabilizing Compound is in the family of Serine proteases which are further classified into clans of proteases or otherwise known as a group of enzymes whose catalytic function is, to breakdown the bonds of proteins. The identified clan for fibrin stabilizing compound is classified as chymotrypsin type of amino acids that is contains a side-chain molecule specific to each. These proteases are directly involved in chemical reactions or catalysis. They function as a general acid-base, electrophilic or nucleophilic catalysts and polarize and stabilize the transition state of binding interactions. However the facilitated catalysis reaction remains a mystery.

Serine Proteases are probably the most thoroughly investigated enzyme system. Catalysis and specificity are not simply controlled by a few residues, but are rather a property of the entire protein framework, controlled via the distribution of charge across a network of hydrogen bonds and perhaps also by the coupling of domain motion to the chemical transformation. Fibrinogen, Factor V Factor VIII and Factor XIII (FXIIII) contain neutral or positively charged residues that influence binding specificity. This binding capability allows for other like molecules to potentially form linkages.

In a preferred embodiment, Rats were pre-dosed with 20 mg/kg TNBS to induce ulcerative colitis. Once induced, rats were treated with either vehicle control or FXIII. Rats showed a dose response for FXIII levels in serum. 80 U/Kg group showed statistically significant improvement in histological colon scores from vehicle control at 10 days. 40 U/Kg group showed a trend towards significance for the same parameter. There was also an indication of re-epithelialization (healing) in two treated (one at 80 U/Kg and one at 160 U/Kg) rats.

These results confirm that moderate to severe ulcerative colitis in animals is accompanied by an acquired deficiency of plasma FXIII. Further, this acquired deficiency is mechanistically relevant to the disease state. Finally, therapeutic replacement of plasma FXIII results in amelioration of the disease that can be measured quantitatively through the microscopic evaluation of mucosal tissue in the colon.

All numerical ranges herein include all numerical values and ranges of all numerical values within the recited numerical ranges. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the Exhibit (page 5).

### REFERENCES

Buchacher, A. & Iberer, G. (2006). Purification of intravenous immunoglobulin G from human plasma—aspects of yield and virus safety. Biotechnol. J. 1, 148-163.
Hahn, R., Bauerhansl, P., Shimahara, K., Wizniewski, C., Tscheliessnig, A. & Jungbauer, A.(2005). Comparison of protein A affinity sorbents II. Mass transfer properties. J.Chromatogr. A 1093, 98-110.
Gianazza, E. & Arnaud, P. (1982). A general method for fractionation of plasma proteins. Dye-ligand affinity chromatography on immobilized Cibacron blue F3-GA. Biochem. J. 201, 129-136.
Rath, V. L.; Verdugo, D.; Hemmerich, S. Sulfotransferase structural biology and inhibitor discovery. Drug DiscoVery Today 2004, 9, 1003-1011.
Koehn, F. E. Therapeutic potential of natural product signal transduction agents. Curr. Opin. Biotechnol. 2006, 17, 631-637.
Koch, M. A.; Waldmann, H. Protein structure similarity clustering and natural product structure as guiding principles in drug discovery. Drug DiscoVery Today 2005, 10, 471-483.
Arve, L.; Voigt, T.; Waldmann, H. Charting biological and chemical space: PSSC and SCONP as guiding principles for the development of compound collections based on natural product scaffolds. QSAR Comb. Sci. 2006, 25, 449-456.
Kornbluth, A., Sachar, D., Ulcerative Colitis Practice Guidelines in Adults (Update): American College of Gastroenterology, American Journal of Gastroenterology 2004 99: 1371
Van Assche, G., Vermeire S., Rutgeerts, P., Treatment Of Severe Steroid Refractory Ulcerative Colitis World J Gastroenterol 2008 Sep. 28; 14(36):5508-5511
D'Argenio, G., Iovino, P., et.al. Factor XIII Improves Gastric Stress Lesions in Rats Digestion 2001;63:220-228 (DOI: 10.1159/000051893)
D'Argenio, G., Cosenza, V., et.al. Butyrate, Mesalamine, and Factor XIII in experimental colitis in the rat: effects of transglutamase activity, Gastroenterology, 1994 February;106 (2):399-404 7905450
D'Argenio, G., Recombinant Factor XIII improves established experimental colitis in rats, Dig Dis Sci. 2000 May ;45 (5):987-97 10795765
Chiaranti, E., Valanzano, R., et.al., Hemostatic abnormalities in inflammatory bowel disease, Thrombosis Research, 1996 Apr. 15;82 (2):137-46 9163067
Seitz, R., Leugner, F., et.al., Ulcerative colitis and crohn's disease: factor XIII, inflammation and haemostasis, Digestion. 1994; 55 (6):361-7 7705548
Chamouard, P., Grunebaum, L., et.al., Significance of diminished factor XIII in Crohn's disease, Am J Gastroenterol. 1998 April;93 (4):610-4 9576457
Linskens, R. K., A van Bodergraven, A., Predictive value of inflammatory and coagulation parameters in the course of severe ulcerative colitis, Dig Dis Sci. 2001 March;46 (3):644-8 11318546
Suzuki, R., Toda, H., Takamura, Y., Dynamics of blood coagulation factor XIII in ulcerative colitis and preliminary study of the factor XIII concentrate, Blut. 1989 August;59 (2):162-4 2765674
Lorenz, R., Olbert, P., Born, P., Factor XIII in chronic inflammatory bowel diseases, Semin Thromb Hemost. 1996 ;22 (5):451-5 8989830
Lorenz, R., Born, P., Classen, M., Substitution of Factor XIII concentrate in treatment refractory ulcerative colitis. A. prospective pilot study, Med Klin (Munich). 1994 Oct. 15;89 (10):534-7 7741858
Lorenz, I L, Heinmuller, M., et.al., Substitution of Factor XIII: a therapeutic approach to ulcerative colitis, Haemostasis. 1991 ;21:5-9 1677916
Lashner B., Inflammatory Bowel Disease, The Cleveland Clinic Disease Management Project, Copyright 2003 The Cleveland Clinic Foundation
Loftus, E V Jr1; Silverstein, M D3; Sandborn, W J1; Tremaine, W J1; Harmsen, S2; Zinmeister, A R., Gut, Volume 46(3), 1 Mar. 2000, pp 336-343
Stonnington, C., Phillips S., Melton L., Zinmeister A., Chronic Ulcerative Colitis: Incidence and Prevalence in a Community, Gut 1987; 28, 402-409

What is claimed is:

1. A method of optimizing therapeutic efficacy for the treatment of Inflammatory Bowel Disease (IDB), comprising:
a. Administering a series of engineered fibrin-stabilizing composition deliveries to a subject having said ulcerative colitis;
b. Where said injections spread over a period of 1 to 10 days;
c. Where injections are performed daily;
d. And individual injections are in the range of 1250 to 3000 concentrate of blood units.

2. A method such as claim 1 where delivery is intravenous, direct placement or means of administration.

3. A method such as claim 1 where IBD is ulcerative colitis.

4. A method such as claim 1 where IBD is Crohn's disease.

5. A method such as claim 1 where IBD is infectious colitis.

6. A method such as claims 1 to 5 where engineered fibrin-stabilizing composition consists of
a. Factor XIII;
b. One or more elements from the following: Ligands, fibrinogen, thromoplastin.

7. A method such as claims 1 to 5 where engineered fibrin-stabilizing composition is realized using an elution process such as Iodid (see item 8) or produces a complex consisting of Iodid-Fibrin Stabilizing Molecule.

8. A method such as claim 6 where said ligands is among: Iodid, bromide, sulfide, thiocyanate, chloride, nitrate, azide, fluoride, hydroxide, oxalate, water, nitrite, isothiocyanate, acetonitrile, sulfite, ammonia, ethylenedianmine, 1,10-phenanthroline, nitrate, thriphenylphosphone, carbon monoxide, acetylacetonate, akene, benzene, 1,2-Bid(diphenylphosphon)ethane, collores, crown ethers, 2,2,-crypt, cryptate, cyclopentadienyl, diethylenetriamine, dimethylglyoximate, ethylendiaminnetriacetate, glycinate, hem, nitrosyl, oxo, pirazine, 2,2′,5′,2-terpyridine, trizacyclononane, tricyclihexylphosphine, triethylenetetramine, trimephosphine, tri(o-totyl)phosphine, tris(2-aminoethyl)amine, tris(2-diphenylphosphineethile)amine, terpyridine, tropylium.

9. A fibrin-stabilizing composition consisting of:
a. Factor XIII;
b. One or more elements from the following: Ligands, fibrinogen, thromoplastin.

10. A fibrin-stabilizing composition such as claim 9 where ligands is among: Iodid, bromide, sulfide, thiocyanate, chloride, nitrate, azide, fluoride, hydroxide, oxalate, water, nitrite, isothiocyanate, acetonitrile, sulfite, ammonia, ethylenedianmine, 2,2′-bipyridine, 1,10-phenanthroline, nitrate, thriphenylphosphone, carbon monoxide, acetylacetonate, akene, benzene, 1,2-Bid(diphenylphosphon)ethane, collores, crown ethers, 2,2,-crypt, cryptate, cyclopentadienyl, diethylenetriamine, dimethylglyoximate, ethylendiaminnetriacetate, glycinate, hem, nitrosyl, oxo, pirazine, 2,2′,5′,2-terpyridine, trizacyclononane, tricyclihexylphosphine, triethylenetetramine, trimephosphine, tri(o-totyl)phosphine, tris(2-aminoethyl)amine, tris (2-diphenylphosphineethile)amine, terpyridine, tropylium.

11. A fibrin-stabilizing composition including a ligand:
a. With affinity for epithelium lining;
b. Miscible with Factor III.
